(19) United States
(12) Patent Application Publication
(10) Pub. No.: US 2013/0104914 A1
(43) Pub. Date: May 2, 2013

(54) Bra accessory to minimize discomfort, rash, and infection

(71) Applicant: Judith Quale Stewart, Davidson, NC (US)

(72) Inventor: Judith Quale Stewart, Davidson, NC (US)

(21) Appl. No.: 13/317,909

(22) Filed: Nov. 1, 2011

(51) Int. Cl. A61F 13/14; A61F 13/45; A41D 27/26

(52) U.S. Cl. 128/889; 2/267; 604/385.01

(57) ABSTRACT

Disclosed is a bra accessory for eliminating or substantially reducing the discomfort, chaffing, pain, rash, intertrigo, and infections caused by pressure of a woman's bra cup rim against her chest, and by perspiration trapped between the woman's breast and chest.

### CROSS REFERENCE TO RELATED APPLICATIONS

None

### FEDERALLY SPONSORED RESEARCH

Not applicable.

### SEQUENCE LISTING OR PROGRAM

Not applicable

### BACKGROUND

1. Field

The present invention is in the field of women's personal items. More specifically, it is a means to distribute over a greater area the pressure of the rim of a bra pressing against a woman's chest, and to absorb perspiration harbored between the breast and the chest, thereby relieving and preventing chafing, pain, discomfort, rash, intertrigo and infections.

2. Prior Art

Absorption of perspiration by various pads and materials is an old art. Abrahamson, in his 1930 U.S. Pat. No. 1,750,084 illustrates a band that separates the breast from the chest, thereby eliminating friction and chafing, and absorbs perspiration between the skin of the breast and the skin of the chest.

Several other patents cover sanitary pads, tampons, and absorbent underarm perspiration pads, all of which are of interest for their materials because of their absorbency function, but do not bear directly on the main physical features of the present invention

Hartman, in her 1995 U.S. Pat. No. 5,603,653 illustrates a pad that absorbs perspiration between the breast and the chest.

Gavitt and Ferrer reveal in their patent application 20080004588 a pair of pads that absorb perspiration between the breasts and the chest.

However, neither Abrahamson's, Hartman's, nor Gavitt's and Ferrer's patent applications provide a means of relieving discomfort caused by pressure against the chest from the lower rim Judith Q. Stewart and Jack R. Lander, filing pro se of the bra, particularly when such bra has an under-wire, and the typical wearer's breasts are large, and therefore heavy.

None of the four prior art citations above discloses the combination we disclose herein. The problem we address is more than irritation caused by perspiration, as stated in the Hartman, and Gavitt and Ferrer applications. The present invention dramatically reduces or eliminates pain and discomfort caused by the relatively small area of a traditional bra cup's lower rim pressing against the wearer's chest. The weight of breasts, especially large, sagging breasts, and the resulting vector delivered against the rims of the bra cups supporting them, plus the tension from the girding effect of the bra itself, causes discomfort and pain such that many women prefer the disadvantages of not wearing a bra much of the time, or even at all.

Bras that are equipped with under-wires or similar stiff reinforcement exacerbate such discomfort and pain. The typical under-wire does not conform closely to the lateral curvature of women's chests, thereby resulting in pressure points, especially against the wearer's ribs and scars from surgery.

Bra padding, for the purpose of augmenting the size and enhancing the shape of the breasts, is well understood and practiced. However, such padding does not serve to relieve discomfort, or absorb perspiration that is often trapped between the breast and the chest. Indeed, relatively heavy padding, such as silicone, may add weight, and exacerbate discomfort and pain.

The common name for the soreness and disease that is caused by perspiration and pressure from breasts pressing against the chest is intertrigo. This is not a condition exclusive to older women, or merely women with sagging breasts; women with average breasts may also suffer from this condition if their breasts lie in contact with their chests at any point. Women who have had breast surgery or open-heart surgery are especially prone to pain and to intertrigo and its complications, which can be severe. Breast cancer patients are especially susceptible to the irritation, chaffing, rash, pain, intertrigo, and yeast infections.

Although intertrigo is a common and often painful condition, surgical breast reduction or augmentation, breast surgery due to cancer or open-heart surgery are, in many cases, the most painful of conditions associated with pressure from the breasts supported or unsupported by bras. Adhesions from surgery form under scars, and are especially aggravated by pressure from the bra rim and perspiration, and cause great suffering.

To relieve the discomfort and pain from the aforementioned conditions, the present invention provides padding between the lower part of the breast and the chest. Such padding lifts the breast up and away from the chest, and transfers part of its weight to a substantially arcuate buffer panel, which, in turn, is attached or adhered to said padding that conforms to the substantially arcuate contour of the bra's lower rim. The arcuate buffer panel extends above and below that rim. Depending on the physical characteristics of the wearer's breasts, and the wearer's objectives, the padding can be increased or decreased.

The buffer panel joined to the padding is made of a material that is stiff relative to the padding. The effective area of the buffer panel is substantially greater than the chest contact area of the lower rim of the typical bra cup. In one embodiment, the buffer panel extends about one-quarter of an inch above and below said rim for the entire curvature of the buffer panel, thereby reducing the pressure per unit of area delivered by the bra's lower rim without our bra accessory, and thereby substantially eliminating the discomfort and pain of the wearer.

Various materials can be used to produce the buffer panels and pads, and a feature of the present invention is to employ inherently permeable materials, or materials that are modified to create permeability, in order to disperse, transport, absorb, and hold the perspiration. In practice, the pad and the buffer panel may be covered with cloth or a cloth-like material to aid in dispersing perspiration before transporting it through the buffer panel to the pad. This covering will provide added comfort, and prevent the interstitial spaces from accidentally pinching the wearer if the wearer should move in a way that would tend to compress the bra accessory. The buffer panels may be made from a permeable material, or may contain conduits, such as slits or holes, in a non-permeable material to promote transport of perspiration from the chest to the pad.

### SUMMARY OF INVENTION

Our bra accessory comprises a buffer panel joined to a pad, whereby this combination substantially reduces pressure on a woman's chest, such that without our bra accessory is concentrated on a relatively small area of the wearer's chest, due to the relatively small area of the bra's lower rim, causing pain or discomfort. Our bra accessory also separates the breast from the chest in those cases where pendulous contact would otherwise occur. And our bra accessory absorbs perspiration that, without our bra accessory, may be harbored between the breast and the chest causing pain, discomfort, rash, intertrigo, and infection in the area between the breast and the chest.

### BRIEF DESCRIPTION OF DRAWINGS

FIG. 1 is a front view of the bra accessory.

FIG. 2 is a cross-sectional view of the bra accessory with uncovered triangular shaped pad.

FIG. 3 is a cross-sectional view of a bra cup in place with a large, non-sagging breast.

FIG. 4 is a cross-sectional view of a bra cup in place with a large, sagging breast showing the perspiration-trapping interface of said breast with the wearer's chest.

FIG. 5 is a cross-sectional view of a bra cup with bra accessory in place.

FIG. 6 is cross-sectional view of bra accessory with cloth or cloth-like covering.

FIG. 7 is a front view of the bra buffer panel showing interstitial spaces and perspiration conduit holes.

FIG. 8 is a cross-sectional view of the bra accessory with flat shaped pad.

FIG. 9 is a cross-sectional view of the bra accessory with flat shaped pad in place in the bra cup.

### DETAILED DESCRIPTION

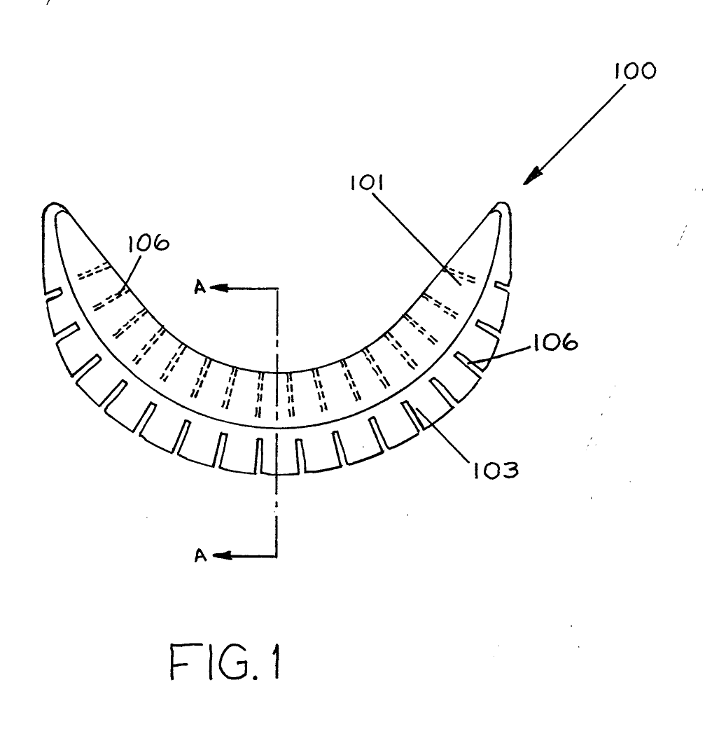

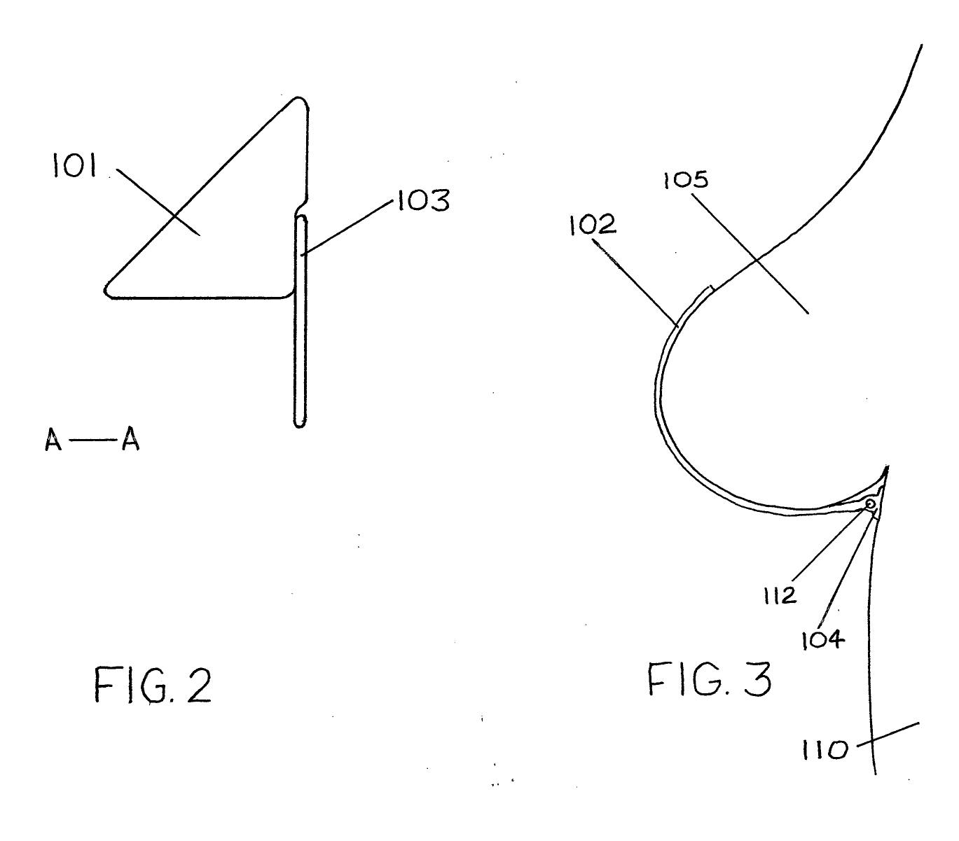

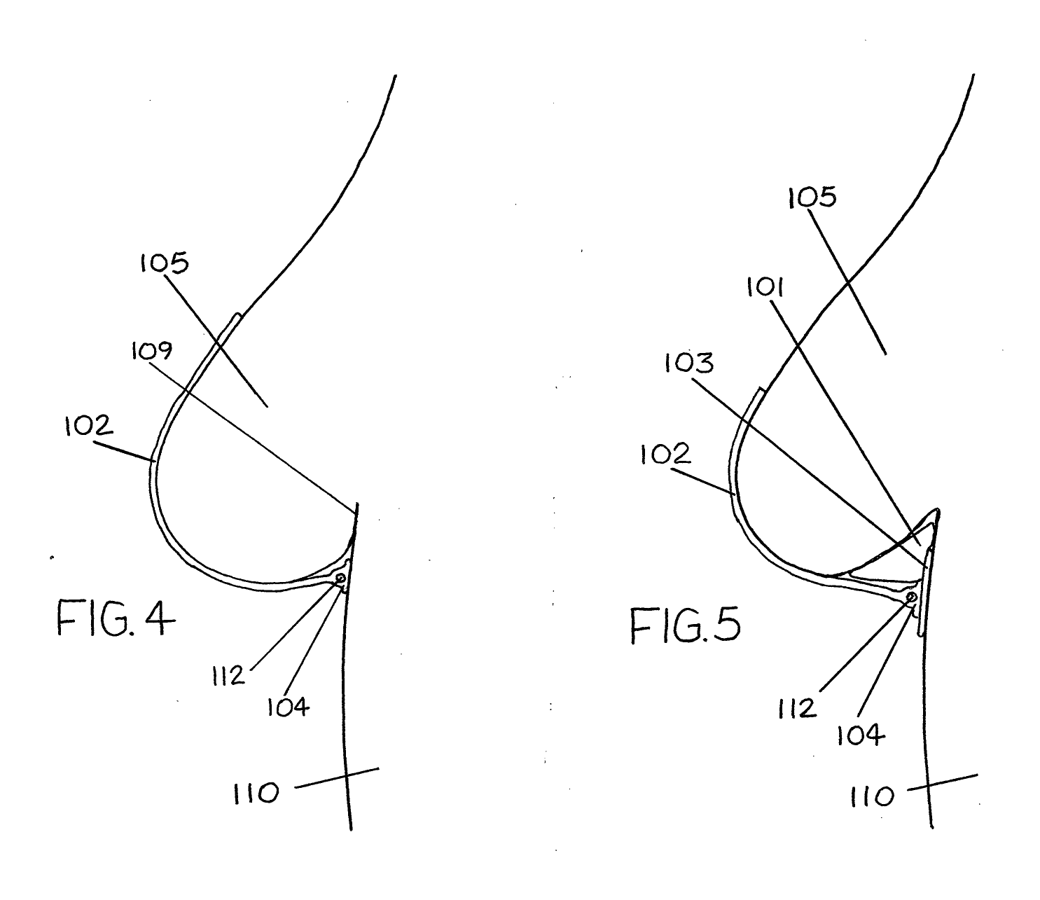

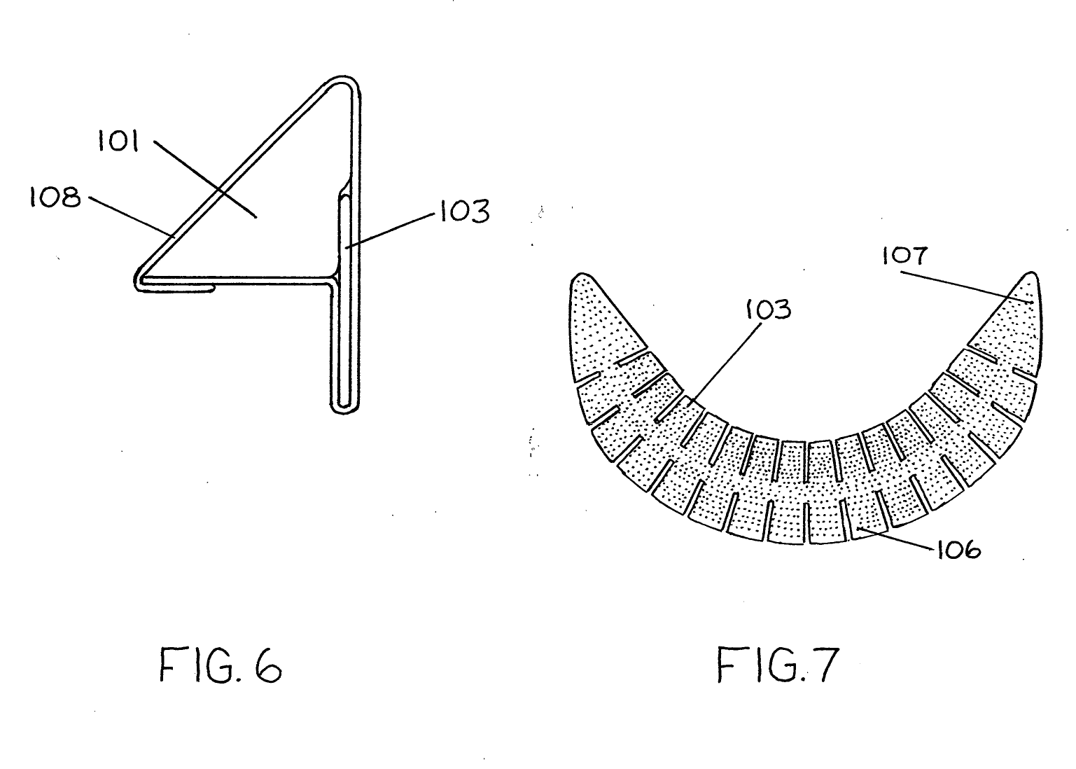

### FIGS. 1 through 7—of FIRST EMBODIMENT

In use, pad 101 of bra accessory 100 is placed in the lower part of bra cup 102 with buffer panel 103 positioned next to and outside of lower rim 104 of bra cup 102. Bra is put on by the wearer in the usual manner, making sure that the lower part of buffer panel 103 extends below the lower rim 104 of bra cup 102.

Pain or discomfort is relieved by delivering the pressure caused by breast 105′s weight to lower rim 104 of bra cup 102, and thence to buffer panel 103 which spreads the pressure load across the area of buffer panel 103, said area being substantially greater than the area of lower rim 104 of bra cup 102, thereby reducing pressure per unit of area that bears on chest 110. Perspiration that forms between chest 110 and buffer panel 103 is transported to pad 101 through permeable material of buffer panel 103 or through conduit holes 107. Discomfort, pain, rash, intertrigo, and disease at breast/chest interface 109 from perspiration and movement is avoided due to breast 105 being held away from chest 110 by pad 101, which also directly absorbs perspiration from breast 105 and chest 110.

Buffer panel 103 is made with interstitial spaces 106 to allow displacement spacing for forming into various curve sizes and shapes to fit lower rim 104 of bra cup 102 thereby accommodating various breast shapes, and bra cup sizes. Buffer panel 103 is constructed of a formable, relatively non-brittle polymer, such as 0.060 thick polycarbonate, which, together with interstitial spaces 106, is sufficiently flexible and formable to allow lateral conformance with the shape of the wearer's chest, and yet is sufficiently stiff in the vertical direction to prevent significant deformation due to pressure from lower rim 104 of bra cup 102. Buffer panel 103 can be formed by bending beyond the yield point of its material. Thus easily formable aluminum, such as #1100 or #3003, mild steel, and formable plastics such as polycarbonate, and the like, offer a variety of hypoallergenic materials that can be used to advantage for buffer panel 103. We must emphasize the non-isotropic nature of buffer panel 103, having inherent stiffness of its material in the vertical direction, while, due to its interstitial slits, having substantially less stiffness in the lateral direction, thereby more easily conforming to the bra wearer's chest.

Pad 101 is made from a permeable material such as used in disposable diapers, tampons, etc., and is joined to buffer panel 103 by any of several means such as sewing, hook and loop, mechanical snap, adhesive, slides, pins, and the like.

### FIGS. 1 through 7—Second Embodiment

A second embodiment of bra accessory 100 covers buffer panel 103 and pad 101 with permeable cover 108. Bra accessory 100 is placed in the lower part of bra cup 102 with buffer panel 103 contiguous to and outside of bra cup lower rim 104. Permeable cover 108 on chest side of buffer panel 103 disperses perspiration, and transports it efficiently through perspiration conduit holes 107, and thence to pad 101 where it is accumulated and contained. Permeable cover 108 may be made from other materials such as a non-woven cloth, sponge, flexible foam, and the like. Whatever material is used to cover buffer panel 103 and pad 101, it must be sufficiently flexible so as to allow its forming, along with buffer panel 103, to fit the curvature of bra cup 102.

### FIGS. 1 through 7—Third Embodiment

A third embodiment of bra accessory 100 is substantially the same as that of the second embodiment except that buffer panel 103 is made from an inherently permeable material, and may include slits to aid in lateral flexibility.

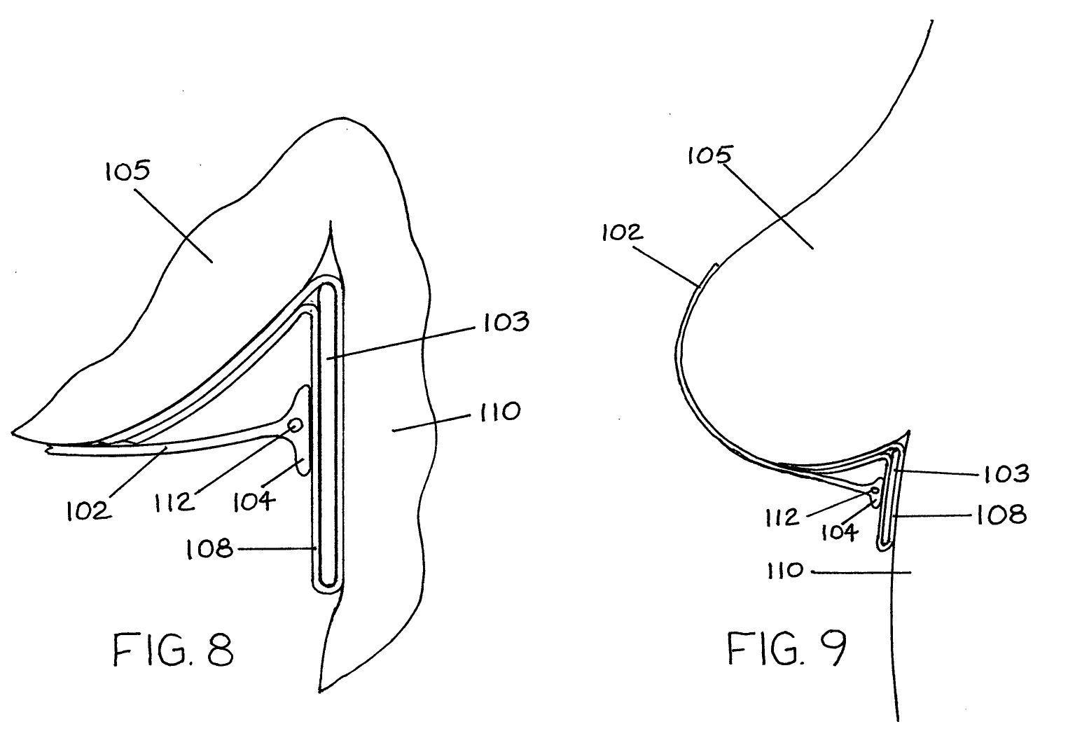

### FIGS. 8 and 9—Fourth Embodiment

A fourth embodiment of bra accessory 100 discloses a flat pad 113 which acts to hold buffer panel 103 in place contiguous to bra cup 102, and absorbs perspiration, but does not provide a substantial partition between a pendulous breast and the chest. This embodiment may be used by women who do not suffer from the irritation, rash, and disease caused by entrapment of perspiration between the breast and chest. However, pain and discomfort is relieved by delivering the pressure caused by breast 105′s weight to bra cup lower rim 104, and thence to buffer panel 103 which spreads the pressure load across the area of buffer panel 103, said area being substantially greater than the area of bra cup lower rim 104, thereby reducing pressure per unit of area that would otherwise bear on chest 110, and thereby reduce discomfort and pain.

### Of Fifth Embodiment

A fifth embodiment of bra accessory 100 is that of buffer panel 103c absent interstitial spaces 106, and made in a variety of sizes and lateral curvatures that, without reforming or adjustment, fit various bra cup sizes without the need for interstitial spaces 106.

### Conclusions, Ramifications, and Scope

Accordingly, the reader will see that the essence of the present invention derives from the basis of a selectively flexible buffer panel that can be bent to conform to the wearer's bra and chest; provides padding that directs the breast away from the chest to absorb perspiration, and prevent trapped perspiration; spreads out the pressure bearing against the bra wearer's chest from the bra cup's lower rim over a greater area than that afforded by a typical bra, thereby diminishing the pressure per unit of area; consequently eliminating or substantially reducing pain and discomfort, and avoiding rashes and infections.

Although we have described the present invention as a bra accessory, it should be understood that the present invention, itself, or elements of the present invention, could easily be incorporated in a bra at the time of the bra's manufacture, thereby providing a novel product to the market based on the present invention. Furthermore, a wide range of materials are readily available for designing and producing our women's bra accessory, and the materials, shapes, sizes, and features of the present invention can be changed without changing the objectives, benefits, and spirit of the present invention.

We claim:

1. A bra accessory comprising:
a) an arcuate buffer panel, substantially greater in area than the chest contact area of the lower rim of a typical bra cup, positioned between said lower rim of said bra cup and the chest of a bra wearer,
b) a pad,
c) a means of joining said pad to said arcuate buffer panel.

2. The bra accessory of claim 1 wherein said arcuate buffer panel has a plurality of interstitial slits of predetermined width and length along its edges, thereby providing space whereby the material lying on either side of said interstitial slits may displace into said interstitial slits to accommodate the reforming by hand of the arcuate buffer panel in order to substantially conform to the arcuate curvature of said lower rim of said bra cup, and to the lateral curvature of said bra wearer's said chest.

3. The bra accessory of claim 1 wherein said arcuate buffer panel is permeable.

4. The bra accessory of claim 1 wherein said arcuate buffer panel is stiff in the vertical direction due to the inherent stiffness of the material from which it is made, and is flexible in the lateral direction due to the number of, and dimensions of, said interstitial slits.

5. The bra accessory of claim 1 wherein said pad is shaped to conform to said arcuate buffer panel.

6. The bra accessory of claim 1 wherein said means of joining said pad to said arcuate buffer panel are conveniently separable.

7. The bra accessory of claim 1 wherein said pad is made of an absorbent, hypoallergenic material.

8. The bra accessory of claim 1 wherein the cross-section of said pad is flat.

9. The bra accessory of claim 1 wherein the cross-section of said pad is triangular or wedge shape.

10. The bra accessory according to claim 9 wherein a portion of said pad is positioned between, and in direct contact with, said breast of said bra wearer and said chest of said bra wearer, whereby perspiration from said breast and said chest are absorbed directly into said pad, thereby eliminating or substantially reducing chafing, discomfort, pain, rash, intertrigo, and infections.

11. The bra accessory according to claim 9 wherein perspiration is further absorbed from said chest of said bra wearer through a permeable cover at covers said buffer panel at least on the side of said buffer panel that faces said bra wearer's said chest, thence through said buffer panel into said pad, thereby eliminating or substantially reducing chafing, discomfort, pain, rash, intertrigo, and infections.

12. The bra accessory according to claim 1 wherein the material of said pad is selected from the group of hypoallergenic materials consisting of natural fibers, man-made fibers, natural sponge, man-made sponge, elastomeric foam, and polymeric foam.

13. The bra accessory according to claim 1 wherein the material of said buffer panel is selected from the group consisting of a permeable material, or a non-permeable material,

14. The bra accessory according to claim 1 wherein said buffer panel is covered, on the side of said buffer panel that is toward said bra wearer's chest, with a permeable hypoallergenic material selected from a group consisting of paper, woven cloth, non-woven cloth, natural sponge, man-made sponge, and elastomeric foam,

14. The bra accessory according to claim 1 wherein said buffer panel is configured with holes of a predetermined size so as to be permeable, thereby permitting transfer of perspiration from said chest of said bra wearer to pass through said buffer panel into said pad, whereby chafing, discomfort, pain, rash, intertrigo, and infections are eliminated or substantially reduced.

12. The bra accessory according to claim 1 wherein material of said buffer panel material is further selected from the group of formably compliant materials comprising aluminum, steel, polycarbonate polymer, and the like.

13. The bra accessory according to claim wherein said buffer panel and said pad are enveloped together in a material selected from the group comprising woven cloth, unwoven cloth-like material, sponge, elastomeric foam, and the like.

16. The bra accessory according to claim that is disposable after wearing a predetermined number of times.
